SEQUENCE LISTING (16 sequences)

SEQ ID NO: 1 - protein, 354 aa, synthetic construct
MKKNGRDNNFYHLYKNKYLITGVTILSFILMFQYKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 2 - DNA, 1065 bp, synthetic construct
  source 1..1065: mol_type = other DNA
atgaagaagaatggaagagacaacaacttctaccacttgtacaagaacaagtacctgatcacgggtgtgacaatcctgtccttcatcctcatgtttcaatacaagtaccatgaagtgctaaccctacacgataatagtgaaaatgctgtacagagcagtaagcttttctgggcgcgactcctcttcggacgaacaaggagtcgaattacagggcaaatattaaaaatggaaatcccaaacacatacagattgttcattttcaattttttaattaaatacatgcacatcaataaggaagaaataaaatacccaatagagtcttacaaatccatcggagattttttctcccgttatattagagaggaaacgagacccattggagatgttagtgattactctatagtcagtccatgtgacagtgaactcatagattacggagaattaacctcagaatatctagaaaatattaagggagtcaaatttaatgtaaacactttcttgggatccaaattccagaagaagcataatgatggaagtaccaaatttttttatgccattttttatttaagtccaaaaaaataccaccattttcatgccccttttaatttcaagtacaaaattaggagacacatatctggagaattatttccagtttttcaaggcatgtttaaatttattaacaacctctttaatattaacgagagggtaatcctgtccggggaatggaaaggtggcaatgtgtattatgccgccattagtgcttacaatgtaggaaatattaaaattattaatgatgaagaattggttacgaataatttaaggcatcagttaagctacatgggaggagatatcaacaccaagattttcgactcctataaaagtgtcgaagttggagacgaaattggggaattcagaatgggctcatccattgttgtaatttttgaaaataaaaaggacttctcctggaatgtcaaccaaaatcaaactgtttccgtaggccagaggcttggcgggatcggtgaacccgtcaaggaggaaaacaggttcatcaaaattagaagctga

SEQ ID NO: 3 - protein, 321 aa, synthetic construct
MKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 4 - protein, 321 aa, synthetic construct
MKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFHVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 5 - protein, 321 aa, synthetic construct
MKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVNQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 6 - protein, 603 aa, synthetic construct
MCHQQLVISWFSLVFLASPLVAMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSGSGSGSDYKDDDDKGGSSMKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 7 - protein, 603 aa, synthetic construct
MCHQQLVISWFSLVFLASPLVAMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSGSGSGSDYKDDDDKGGSSMKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFHVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 8 - protein, 603 aa, synthetic construct
MCHQQLVISWFSLVFLASPLVAMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSGSGSGSDYKDDDDKGGSSMKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVNQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 9 - DNA, 966 bp, synthetic construct
  source 1..966: mol_type = other DNA
atgaaataccacgaagtcctcaccctccatgacaactcagaaaacgccgtccagtcttcaaaactgttctgggctaggctgttgttcggccgcacgagaagtcggatcactggccagatattgaaaatggaaataccgaatacctataggttgtttattttcaatttcctgatcaaatacatgcacatcaataaggaggagatcaaataccctatcgaatcatataaaagcatcggcgactttttttctaggtacattagagaagaaacacggccaattggtgacgtcagcgattacagtatcgtgagcccttgcgatagcgagttgattgactatggtgaactcacaagcgaatacctcgaaaatatcaaaggcgtgaaattcaacgttaacacctttctggggtcaaaatttcaaaagaagcacaacgatggtagcacgaaattcttctatgctatcttctacttgtcccccaagaagtatcaccatttccatgctccatttaacttcaaatataagatccggagacacatatctggcgaactcttccccgttttccaaggcatgtttaagtttatcaataacctgttcaatattaacgagagagtgatacttagtggggagtggaaggggggtaatgtttattatgctgctattagcgcctacaatgttgggaacattaagattatcaatgacgaagagctggtcaccaacaatctgagacaccagctgtcttatatgggcggggacattaacacaaagatctttgattcttataagagcgttgaagtaggggacgagattggcgagttcaggatgggctcttcaattgtggtaatcttcgaaaacaaaaaggacttttcctggaatgttaaccagaatcaaactgtgagtgtaggacaaagattgggagggatcggggagcctgtgaaggaagagaaccgcttcattaagatccgctcctag

SEQ ID NO: 10 - protein, 409 aa, synthetic construct
MATSVGHRCLGLLHGVAPWRSSLHPCEITALSQSLQPLRKLPFRAFRTDARKIHTAPARTMFLLRPLPILLVTGGGYAGYRQYEKYRERELEKLGLEIPPKLAGHWEVALYKSVPTRLLSRAWGRLNQVELPHWLRRPVYSLYIWTFGVNMKEAAVEDLHHYRNLSEFFRRKLKPQARPVCGLHSVISPSDGRILNFGQVKNCEVEQVKGVTYSLESFLGPRMCTEDLPFPPAASCDSFKNQLVTREGNELYHCVIYLAPGDYHCFHSPTDWTVSHRRHFPGSLMSVNPGMARWIKELFCHNERVVLTGDWKHGFFSLTAVGATNVGSIRIYFDRDLHTNSPRHSKGSYNDFSFVTHTNREGVPMRKGEHLGEFNLGSTIVLIFEAPKDFNFQLKTGQKIRFGEALGSL

SEQ ID NO: 11 - protein, 406 aa, synthetic construct
MAASGGRACVRSLRGGVLWRSSPCHYESTATRHFLGTLQKLPLQAGVRNFHTAPVRSLFLLRPVPILLATGGGYAGYRQYEKYRERKLEKLGLEIPPKLASHWEVSLYKSVPTRLLSRACGRLNQVELPYWLRRPVYSLYIWTFGVNMTEAAVEDLHHYRNLSEFFRRKLKPQARPVCGLHCVTSPSDGKILTFGQVKNSEVEQVKGVTYSLESFLGPRANTEDLPFPPASSSDSFRNQLVTREGNELYHCVIYLAPGDYHCFHSPTDWTISHRRHFPGSLMSVNPGMARWIKELFCHNERVVLTGDWKHGFFSLTAVGATNVGSIRIHFDRDLHTNSPRYSKGSYNDLSFVTHANKEGIPMRKGEPLGEFNLGSTIVLIFEAPKDFNFRLKAGQKIRFGEALGSL

SEQ ID NO: 12 - protein, 322 aa, synthetic construct
MLNSFKLSLQYILPKLWLTRLAGWGASKRAGWLTKLVIDLFVKYYKVDMKEAQKPDTASYRTFNEFFVRPLRDEVRPIDTDPNVLVMPADGVISQLGKIEEDKILQAKGHNYSLEALLAGNYLMADLFRNGTFVTTYLSPRDYHRVHMPCNGILREMIYVPGDLFSVNHLTAQNVPNLFARNERVICLFDTEFGPMAQILVGATIVGSIETVWAGTITPPREGIIKRWTWPAGENDGSVALLKGQEMGRFKLGSTVINLFAPGKVNLVEQLESLSVTKIGQPLAVSTETFVTPDAEPAPLPAEEIEAEHDASPLVDDKKDQV

SEQ ID NO: 13 - protein, 340 aa, synthetic construct
MLVTGGGYAGYRQYEKYRERELEKLGLEIPPKLAGHWEVALYKSVPTRLLSRAWGRLNQVELPHWLRRPVYSLYIWTFGVNMKEAAVEDLHHYRNLSEFFRRKLKPQARPVCGLHSVISPSDGRILNFGQVKNCEVEQVKGVTYSLESFLGPRMCTEDLPFPPAASCDSFKNQLVTREGNELYHCVIYLAPGDYHCFHSPTDWTVSHRRHFPGSLMSVNPGMARWIKELFCHNERVVLTGDWKHGFFSLTAVGATNVGSIRIYFDRDLHTNSPRHSKGSYNDFSFVTHTNREGVPMRKGEHLGEFNLGSTIVLIFEAPKDFNFQLKTGQKIRFGEALGSL

SEQ ID NO: 14 - protein, 316 aa, synthetic construct
MGLEIPPKLASHWEVSLYKSVPTRLLSRACGRLNQVELPYWLRRPVYSLYIWTFGVNMTEAAVEDLHHYRNLSEFFRRKLKPQARPVCGLHCVTSPSDGKILTFGQVKNSEVEQVKGVTYSLESFLGPRANTEDLPFPPASSSDSFRNQLVTREGNELYHCVIYLAPGDYHCFHSPTDWTISHRRHFPGSLMSVNPGMARWIKELFCHNERVVLTGDWKHGFFSLTAVGATNVGSIRIHFDRDLHTNSPRYSKGSYNDLSFVTHANKEGIPMRKGEPLGEFNLGSTIVLIFEAPKDFNFRLKAGQKIRFGEALGSL

SEQ ID NO: 15 - protein, 593 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGMKYHEVLTLHDNSENAVQSSKLFWARLLFGRTRSRITGQILKMEIPNTYRLFIFNFLIKYMHINKEEIKYPIESYKSIGDFFSRYIREETRPIGDVSDYSIVSPCDSELIDYGELTSEYLENIKGVKFNVNTFLGSKFQKKHNDGSTKFFYAIFYLSPKKYHHFHAPFNFKYKIRRHISGELFPVFQGMFKFINNLFNINERVILSGEWKGGNVYYAAISAYNVGNIKIINDEELVTNNLRHQLSYMGGDINTKIFDSYKSVEVGDEIGEFRMGSSIVVIFENKKDFSWNVNQNQTVSVGQRLGGIGEPVKEENRFIKIRS

SEQ ID NO: 16 - protein, 362 aa, synthetic construct
MLSKFYYCKMRKGKSPSSFFSLHKKYLLTGVTILSFIFMFQYKYHEVLTVYEDKTNVQQSSRLFWTRLLFGRTRSRITGRIFNIEIPHSSRLYVYNFFIKYLNINKEEIKYPIESYKSLGDFFSRYIREDTRPIGDLNEYSIVSPCDSEIVDFGELTSNYLDNVKGIKFNIKTFLGSDMIKKYNDDSTSFYYAIFYLSPKKYHHFHAPFNFKYKIRRHISGEVFPVFQGMFKIINNLFDINERVILSGEWKGGHVYYAAISAYNVGNIKIVNDEDLLTNNLRTQLSYMGGDINTKIYDHYKDLEIGDEVGEFKVGSSIIVIFENKKNFKWNVKPNQQISVGERIGGVDQPKQPQNKFIKIRS

(19) United States
(12) Patent Application Publication
(10) Pub. No.: US 2025/0144145 A1
(43) Pub. Date: May 8, 2025

(54) ENGINEERED PSD PROTEIN, EXTRACELLULAR VESICLE, AND PREPARATION METHOD AND USE THEREOF

(71) Applicant: BEIJING ECHO BIOTECH CO., LTD., Beijing (CN)

(72) Inventors: Libo Zhao, Beijing (CN); Jiaxun Zhang, Beijing (CN); Guanyi Kong, Beijing (CN); Hang Zhao, Beijing (CN); Da LIU, Beijing (CN); Zhicheng You, Beijing (CN)

(21) Appl. No.: 18/935,205

(22) Filed: Nov. 1, 2024

(30) Foreign Application Priority Data
Nov. 3, 2023 (CN) 2023114506291

(51) Int. Cl. A61K 35/22; C07K 14/54; C12N 5/071; C12N 9/88

(52) U.S. Cl. CPC A61K 35/22; C07K 14/5434; C12N 5/0686; C12N 9/88; C12Y 401/01065; C07K 2319/21

(57) ABSTRACT

The application discloses an engineered phosphatidylserine decarboxylase (PSD protein) and use thereof, a fusion protein comprising the engineered PSD protein and use thereof, a nucleic acid molecule for encoding the engineered PSD protein or the fusion protein, an expression vector comprising the nucleic acid molecule, an engineered cell comprising the nucleic acid molecule or the expression vector, a method for preparing an engineered extracellular vesicle, an engineered extracellular vesicle and use thereof. In this application, an engineered phosphatidylserine decarboxylase is used for catalyzing phosphatidylserine on the surface of an extracellular vesicle, so that phagocytosis of macrophages is reduced, in-vivo clearance is reduced, and the circulation time of the extracellular vesicle is prolonged.

### TECHNICAL FIELD

The present application belongs to the field of bioengineering, and particularly relates to an engineered PSD protein, an engineered extracellular vesicle, and a preparation method and use thereof.

### BACKGROUND ART

Extracellular vesicles (EVs) are membranous vesicle bodies secreted by cells, with a diameter of approximately 30-1000 nm, which can be taken up by receptor cells. EVs are carriers for the intercellular transport of bio-macromolecules such as proteins, RNA, and lipids. They are important media for cell-to-cell communication, and are therefore considered to be naturally domesticated drug carriers.

The pharmacokinetics of extracellular vesicles is one of the important issues in the development of extracellular vesicle drug delivery systems. It has been reported that, the half-life of intravenously injected extracellular vesicle drugs is less than 10 minutes, and they are mainly cleared by macrophages in the liver (Imai et al., 2015; Morishita et al., 2015). Macrophages mainly recognize phosphatidylserine (PS). The negative charge on the surface of extracellular vesicles caused by phosphatidylserine is recognized by macrophages, and is associated with clearance by macrophages after intravenous injection (Matsumoto et al., 2017).

Currently, it was reported in an article that, Tim4 (a protein specifically binding to phosphatidylserine) beads were used to capture PS(+)-EVs, then reversely screening out PS(−)-EVs, which only account for 10% of natural extracellular vesicles. PS(−)-EVs can greatly prolong the half-life of extracellular vesicles after intravenous injection (Matsumoto et al., 2021). Therefore, how to obtain PS(−)-EVs is a key technology to improve the circulation time of extracellular vesicles.

The commonly used methods in the literatures are: (1) capturing PS(+)-EVs by Tim4-coupled magnetic beads, and then reversely screening out PS(−)-EVs; (2) directly extracting extracellular vesicles from plasma, because the extracellular vesicles in plasma are residual PS(−)-EVs after phagocytosis by macrophages (Matsumoto et al., 2021); (3) purifying phosphatidylserine decarboxylase (PSD) through prokaryotic expression, adding PSD to EV for co-incubation and catalysis in vitro to obtain PS(−)-EVs (Kobayashi et al., 2022).

However, the yield of extracellular vesicles obtained by reverse screening using the Tim4-coupled gel beads is very low, because PS(−)-EVs account for only about 10% of natural extracellular vesicles. The method of extracting PS(−)-EVs from plasma is high in cost and low in yield. The in vitro PSD catalysis method requires the expression and purification of PSD protein, and the PSD protein has low solubility, resulting in low protein yield. Post-expression protein incubation is prone to introducing other contamination. The reaction of in vitro incubation is slow, and long incubation time can easily affect extracellular vesicles. After in vitro incubation, re-purification is required, which increases the extracellular vesicle production process and results in a loss of extracellular vesicle yield.

### SUMMARY

In view of the above problems in the prior art, the present application provides an engineered PSD protein and an engineered extracellular vesicle. The engineered PSD protein catalyzes the phosphatidylserine on the surface of the extracellular vesicles, which reduces the phagocytosis of macrophages, thereby reducing clearance in the body and prolonging the circulation time of the extracellular vesicles.

The particular technical solutions of this application are as follows:
1. An engineered phosphatidylserine decarboxylase (PSD protein) comprising the following three functional domains: X₁X₂X₃X₄(X₅)ₘX₆(X₇)ₙX₈X₉X₁₀ at positions 114-139, X₁₁X₁₂X₁₃X₁₄X₁₅X₁₆X₁₇X₁₈X₁₉X₂₀ at positions 191-120, and X₂₁X₂₂X₂₃ at positions 307-309, referring to the wild-type PSD protein represented by SEQ ID NO: 1;
wherein X₁ and X₂ are each independently Phe, Trp, Leu, Val, Ile or Tyr;
X₄ and X₆ are each independently Arg, Lys, Gln or Asn;
X₈, X₁₁, and X₂₀ are Pro;
X₁₀ is Asp, Glu or Asn;
X₁₄ is Tyr, Trp, Phe, Thr or Ser;
X₁₅, X₁₈, and X₁₉ are each independently His, Asn, Gln, Lys, or Arg;
X₂₁ is Gly;
X₂₂ is Ser;
X₂₃ is Ser, Thr or Val;
X₃, X₅, X₇, X₉, X₁₂, X₁₃, X₁₆, and X₁₇ are each independently any amino acid, m and n respectively represent m and n repeated amino acids, wherein m=1-6, 2-5, 3-4, or any positive integer within such a numerical range, and n=1-12, 2-11, 3-10, 4-9, 5-8, 6-7, or any positive integer within such a numerical range;
preferably, the PSD protein comprises the following three functional domains: FFXRX₆RX₁₂PXD at positions 114-139, PXXYHXXHXP at positions 191-120, and GSS/GST at positions 307-309, referring to the wild-type PSD protein represented by SEQ ID NO: 1, wherein each X is independently any amino acid.
2. The engineered PSD protein according to item 1, which is a PSD protein derived from Plasmodium knowlesi.
3. The engineered PSD protein according to item 1 or 2 is a truncated PSD protein with deletion of amino acids at positions 1-45 from the N-terminus, preferably a truncated PSD protein with deletion of amino acids at positions 1-34 from the N-terminus.
4. The engineered PSD protein according to any one of items 1-3, comprising an insertion, deletion or substitution mutation at one or two positions, wherein the positions are positions 217 and 219 referring to the wild-type PSD protein represented by SEQ ID NO: 1;
preferably, the mutation is a substitution mutation;
preferably, the substitution mutations are P217H and F219N;
preferably, the engineered PSD protein comprises an amino acid sequence selected from any one of SEQ ID NO: 3-5 and SEQ ID NOs: 10-12, or an amino acid sequence having at least 70%, 75%, 80%, 85%, 90%, 91%, 92%, 93%, 94%, 95%, 96%, 97%, 98% or 99% similarity to the amino acid sequence selected from any one of SEQ ID NO: 3-5 and SEQ ID NOs: 10-12.
5. A fusion protein comprising the engineered PSD protein according to any one of items 1-4.
6. The fusion protein according to item 5, further comprising a sequence promoting the solubility of the PSD protein, preferably a GST sequence.
7. The fusion protein according to item 5 or 6, further comprising one or more signal peptides;
preferably, the signal peptide is a signal peptide of a secretory protein;
preferably, the secretory protein is one or more selected from the group consisting of: antibody, cytokine, protein hormone and digestive enzyme;
preferably, the fusion protein comprises an amino acid sequence selected from any one of SEQ ID NOs: 6-8, or an amino acid sequence having at least 70%, 75%, 80%, 85%, 90%, 91%, 92%, 93%, 94%, 95%, 96%, 97%, 98% or 99% similarity to the amino acid sequence selected from any one of SEQ ID NOs: 6-8.
8. A nucleic acid molecule encoding the engineered PSD protein according to any one of items 1-4 or the fusion protein according to any one of items 5-7;
preferably, the sequence of the nucleic acid molecule is represented by SEQ ID NO: 9, or a nucleotide sequence having at least 70%, 75%, 80%, 85%, 90%, 91%, 92%, 93%, 94%, 95%, 96%, 97%, 98% or 99% similarity to SEQ ID NO: 9.
9. An expression vector comprising the nucleic acid molecule according to in item 8.
10. An engineered cell comprising the nucleic acid molecule according to item 8 and a nucleic acid molecule encoding a cargo protein;
preferably, the cargo protein is one or more selected from the group consisting of: a therapeutic peptide, a DNA binding protein, an RNA binding protein, a fluorescent protein, an enzyme, and a linker for connecting to a therapeutic compound;
preferably, the therapeutic peptide is an antibody and/or a cytokine, for example, the cytokine is one or more selected from the group consisting of: a member of the human interleukin family, a member of the tumor necrosis factor family, an interferon, and a T cell engager;
preferably, the RNA binding protein is one or more selected from the group consisting of: L7Ae, hnRNPA2B1, hnRNPC1, hnRNPG, hnRNPK, hnRNPQ, YBX1, HuR, AGO2, IGF2BP1, MEX3C, ANXA2, ALIX, NCL, FUS and MVP.
11. A method for preparing an engineered extracellular vesicle, which comprises the following steps:
culturing the engineered cells according to item 10, and isolating the extracellular vesicle secreted by the engineered cell from a culture medium.
12. An engineered extracellular vesicle comprising the fusion protein according to any one of items 5-7 and a cargo protein;
preferably, the fusion protein is located on the membrane of the extracellular vesicle;
preferably, the cargo protein is one or more selected from the group consisting of: a therapeutic peptide, a DNA binding protein, an RNA binding protein, a fluorescent protein, an enzyme, and a linker for connecting to a therapeutic compound;
preferably, the therapeutic peptide is an antibody and/or a cytokine, for example, the cytokine is one or more selected from the group consisting of: a member of the human interleukin family, a member of the tumor necrosis factor family, an interferon, and a T cell engager;
preferably, the RNA binding protein is one or more selected from the group consisting of: L7Ae, hnRNPA2B1, hnRNPC1, hnRNPG, hnRNPK, hnRNPQ, YBX1, HuR, AGO2, IGF2BP1, MEX3C, ANXA2, ALIX, NCL, FUS and MVP.
13. A pharmaceutical composition comprising the engineered extracellular vesicles according to item 12 or an engineered extracellular vesicle prepared by the method according to item 11, and a pharmaceutically acceptable carrier.
14. Use of the fusion proteins according to any one of items 5-7, the engineered extracellular vesicle according to item 12, or an engineered extracellular vesicle prepared by the method according to item 11 in the preparation of a medicament for diagnosing, treating and/or preventing a disease.
15. A method for diagnosing, treating and/or preventing a disease, comprising administrating to a subject in need thereof an effective amount of the engineered extracellular vesicle according to item 12 or an engineered extracellular vesicle prepared by the method according to item 11.

### Effects of the Application

Different from the low solubility of PSD proteins in most eukaryotic organisms in the prior art, the engineered PSD protein according to the present application naturally exists a part of secretable forms with high solubility and high protein yield. The present application constructs an engineered cell capable of expressing an engineered PSD protein or a fusion protein comprising an engineered PSD protein, so that the engineered PSD protein or fusion protein is secreted into the cell culture supernatant. During the process of cell secretion of extracellular vesicles, PSD-catalyzed carboxyl cleavage of phosphatidylserine can be simultaneously completed. After the engineered extracellular vesicles are extracted, extracellular vesicles with extremely low surface PS content can be obtained, which can significantly reduce macrophage phagocytosis and increase the in vivo circulation time of extracellular vesicles. The method for preparing engineered extracellular vesicles of the present application is simple, time-saving and low-cost, and can also avoid the loss of extracellular vesicles during the re-purification process.

### BRIEF DESCRIPTION OF THE DRAWINGS

FIG. 1A and FIG. 1B are the results for Western blot analysis of PSD cells and HEK293F cells without plasmid transfer.

FIG. 2 shows the identification results for electrophoresis of his-GST-pkPSD_35_319 protein purified by his-affinity. chromatography.

FIG. 3A and FIG. 3B show the results for nano-flow cytometry of the extracellular vesicles in the control group and the extracellular vesicles catalyzed by addition of pkPSD, respectively.

FIG. 4A shows the results for nano-flow cytometry after co-incubating the extracellular vesicles with pkPSD protein at 37° C. for 0 min, 10 min, 30 min, 60 min, and 120 min; FIG. 4B shows the results for nano-flow cytometry after co-incubating the extracellular vesicles with 0 μL, 10 μL, 30 μL, 50 μL, and 70 μL pkPSD protein at 37° C. for 3 h.

FIG. 5A and FIG. 5B are the FlowJo flow cytometry analysis results for the extracellular vesicles 293F EV extracted from HEK293F, extracellular vesicles 293F EV+PSD obtained by catalysis of 293F EV with PSD enzyme, and extracellular vesicles PSD EV extracted based on PSD engineered cells.

FIG. 6 shows the results for nano-flow cytometry after co-incubating 293F EVs in the control group and PSD EVs in the experimental group with RAW264.7 macrophages at different concentrations.

FIG. 7 is a bar graph of the extracellular vesicle content in mouse serum at different time points after the same number of particles of 293F EV (CK EV), PSD (CK EV+PSD) obtained by adding PSD in vitro to catalyze 293F EV, and PSD EV were injected into mice by tail vein.

FIG. 8 shows the detection results for AnV-FITC positive rate/MFI of extracellular vesicles extracted from 293F cells transiently transfected with pkPSD, hPSD, and mPSD.

FIG. 9 shows the results for consistency and similarity analysis of the PSDs of Plasmodium knowlesi, human, mouse, Plasmodium falciparum, and Escherichia coli.

FIG. 10 shows the multiple sequence alignment of PSD proteins from different species.

FIG. 11 shows the results for AnV-FITC positive rate of extracellular vesicles extracted after point mutation of the conservative motif.

FIG. 12 is a schematic diagram of the three-dimensional structure of the protein pocket for the interaction between pkPSD and PS.

FIG. 13 shows the results for AnV-FITC positive rate of mutant pkPSD(P217H), pkPSD(V217H), pkPSD(F219N), pkPSD(Y255T), pkPSD(Y217S, Y255T), and pkPSD(M306L).

FIG. 14 is a schematic plan view of the structures of PS (10:0/10:0) and pkPSD proteins.

FIG. 15 is a bar graph of the extracellular vesicle content in mouse serum at different time points after the same number of particles of 293F EV (CK EV) and pkPSD (P217H) EV were injected into mice by tail vein.

### DETAILED DESCRIPTION

Particular embodiments of the present application will be described in more detail below with reference to the accompanying drawings. Although particular embodiments of the present application are shown in the drawings, it should be understood that the present application can be implemented in various forms and should not be limited to the embodiments set forth herein. On the contrary, these embodiments are provided to enable a more thorough understanding of the present application and to fully convey the scope of the present application to those skilled in the art.

It should be noted that, the words “comprise/include” or “comprising/including” mentioned throughout the specification and claims are open-ended terms, and should be interpreted as “including but not limited to”. The preferred embodiments are described subsequently in the specification for implementing the present application, and the description is for the purpose of illustrating the general principles of the present application and is not intended to limit the scope of the present application. The protection scope of this application shall be determined by the appended claims.

### Definitions

Unless defined otherwise, all technical and scientific terms used herein have the same meanings as understood by one of ordinary skill in the art.

In the present application, the engineered modification can be accomplished by any method known in the art, and many such methods are well known and routine to skilled technicians, such as truncation or fusion of proteins, and substitution, deletion and/or insertion of an amino acid.

The term “extracellular vesicles” (EVs) used herein refers to double-membrane vesicular bodies that are shed from the cell membrane or secreted by cells, with a diameter ranging from 40 nm to 1000 nm. Their main forms are microvesicles (MVs) and exosomes (Exs). Extracellular vesicles are widely present in supernatants of cell culture and various body fluids (blood, lymph, saliva, urine, semen, and milk). They carry a variety of proteins, lipids, DNA, mRNA, miRNA, etc. related to the cell origin, and are involved in processes such as intercellular communication, cell migration, angiogenesis, and immune regulation.

The term “wild type” as used herein has the meaning commonly understood by those skilled in the art, and refers to a typical form of an organism, strain, gene or characteristic that distinguishes it from a mutant or variant when it exists in nature. It can be isolated from resources found in nature, and has not been deliberately modified.

The term “engineered extracellular vesicles” used herein refers to artificially synthesized extracellular vesicles, or extracellular vesicles produced by cells after human intervention, or extracellular vesicles produced by cells after modifying through genetic engineering.

The term “phosphatidylserine” (PS) used herein refers to a glycerophospholipid formed by an ester bond between the phosphate group of phosphatidic acid and the hydroxyl group of serine. It can be converted into phosphatidylcholine and phosphatidylethanolamine. Under normal circumstances, it is located on the inner side of the lipid bilayer of the plasma membrane. During cell apoptosis, it turns inside out, causing a significant increase in the phosphatidylserine on the outer side of the plasma membrane. During the formation process of extracellular vesicles, due to changes in curvature, phosphatidylserine will also appear on the membrane surface.

The term “phosphatidylserine decarboxylase” (PSD, EC 4.1.1.65), also known as PSD protein, used herein refers to an enzyme that catalyzes the cleavage of the carboxyl group of phosphatidylserine to form phosphatidylethanol and carbon dioxide. Other common names include PS decarboxylase and phosphatidyl-L-serine carboxyl lyase. This enzyme is involved in the metabolism of glycine, serine, and threonine, as well as in glycerophospholipid metabolism. It has two cofactors: pyridoxal phosphate and pyruvate. The pkPSD mentioned herein is the PSD derived from the species Plasmodium knowlesi, the hPSD is the PSD from humans (Homo sapiens), and the mPSD is the PSD from mice (Mus musculus).

The term “Annexin A5” (Annexin A5 or Annexin V, AnV) as used herein is a protein in the annexin family. Because it can specifically bind to phosphatidylserine in the presence of calcium ions, it is often used as a non-quantitative probe in flow cytometry for detecting cell apoptosis to detect phosphatidylserine eversion on the cell surface. AnV-FITC is AnV coupled with FITC (fluorescein isothiocyanate) for easy use in Flow cytometry.

The term “similarity” as used herein refers to a percentage calculated by aligning two sequences based on the ratio of the number of identical and similar characters to the alignment length. The alignment algorithms available include BLAST, ClustalW, etc.

As used herein, the term “cargo proteins” refers to proteins carried within extracellular vesicles.

As used herein, the term “signal peptide” refers to a peptide sequence that directs the transport and localization of a protein within a cell, for example, to a certain organelle (such as the endoplasmic reticulum) and/or the cell surface. The signal peptide directs the nascent protein into the endoplasmic reticulum. This is necessary if the receptor is to be glycosylated and anchored in the cell membrane.

As used herein, the term “antibody” refers to an immunoglobulin molecule that specifically binds or is immunoreactive to a particular antigen. Antibodies may include, for example, polyclonal, monoclonal, and genetically engineered antibodies and antigen-binding fragments thereof. The antibody can be, for example, a murine antibody, a chimeric antibody, a humanized antibody, a heteroconjugate antibody, a bispecific antibody, a diabody, a triabody, or a tetrabody.

The term “therapeutic peptide” as used herein refers to a protein or variant thereof having therapeutic activity, including but not limited to: antibodies or antigen-binding fragments thereof, receptors, ligands, cytokines, hormones, and the like.

As used herein, the term “linker for connecting to a therapeutic compound” refers to a substance that attaches a therapeutic compound to a fusion partner.

The term “interleukin” as used herein refers to a class of cytokines produced by and acting on a variety of cells, and it refers to a class of cytokines whose molecular structures and biological functions have been basically clarified, and which are uniformly named for their important regulatory effects. Interleukin belongs to cytokines as well as blood cell growth factor. The two cytokines coordinate and interact with each other to jointly complete the hematopoietic and immune regulation functions. Interleukin plays an important role in transmitting information, activating and regulating immune cells, mediating T and B cell activation, proliferation and differentiation, and in inflammatory responses. Interleukin (IL) is abbreviated as IL. Its function is related to the expression and regulation of immune response. This regulation involves many factors derived from lymphocytes or macrophages. Those derived from lymphocytes include lymphokines, and those derived from macrophages are collectively called monokine. The biological activities of each factor are different (such as macrophage activation, promotion of T cell proliferation, etc.), and the physical and chemical properties of the factors themselves are mostly unclear.

The term “vector” as used herein generally refers to a nucleic acid molecule capable of self-replication in a suitable host, which transfers an inserted nucleic acid molecule into and/or between host cells. The vector may include a vector primarily used to insert DNA or RNA into cells, a vector primarily used to replicate DNA or RNA, and a vector primarily used for expression by transcription and/or translation of DNA or RNA. The vector also includes a vector having multiple functions as described above. The vector may be a polynucleotide that can be transcribed and translated into a polypeptide when introduced into an appropriate host cell. Generally, the vector can produce the desired expression product by culturing appropriate host cells comprising the vector.

The terms “nucleic acid” or “polynucleotide” or “nucleic acid molecule” as used herein generally refer to deoxyribonucleic acid (DNA) or ribonucleic acid (RNA) and polymers thereof in either single- or double-stranded form. Unless specifically limited, the term can include nucleic acids comprising analogs of natural nucleotides that have similar binding properties as a reference nucleic acid (e.g., showing sequence information) and are metabolized in a manner similar to naturally occurring nucleotides. Unless otherwise indicated, the sequence of a nucleic acid may include conservatively modified variants thereof, such as degenerate codon substitutions, alleles, orthologs, SNPs, and complementary sequences, as well as an explicitly indicated sequence.

As used herein, the term “treatment” refers to any intervention that results in any observable beneficial effect of treatment, or any statistically significant indicator of success in treating or ameliorating a disease or condition, such as improving the signs, symptoms, or progression of a disease or pathological condition. For example, a beneficial effect may be demonstrated by a decrease in disease, a delay in the onset of disease or a lessening in severity of clinical symptoms of disease in a subject, a decrease in the frequency with which a subject experiences disease symptoms, a slowing of the progression of disease, a decrease in the number of disease relapses, an improvement in the subject's overall health, or by other parameters specific to a particular disease.

As used herein, the term “prevention” is treatment administrated to a subject who does not show signs of a disease or who shows only early signs of the disease, with the goal of reducing the risk of developing the pathology or further progression of the early disease.

The term “administrate/administrating” as used herein refers to parenteral, intravenous, intraperitoneal, intramuscular, intratumoral, intralesional, intranasal or subcutaneous administration, oral administration, administration by suppository, topical contact, intrathecal administration or implantation of a sustained release device, such as a mini-osmotic pump, to a subject in need thereof.

The term “a subject in need thereof” as used herein refers to an individual who is at risk of or suffering from a disease, disorder or condition, and who can be treated or improved by the pharmaceutical composition or engineered extracellular vesicles described herein.

The terms “effective amount” or “effective dose” as used herein refer to an amount of a pharmaceutical composition or an engineered extracellular vesicle sufficient to achieve a desired (e.g., beneficial) effect in a subject treated with the pharmaceutical composition or the engineered extracellular vesicle, such as an amount sufficient to improve one or more symptoms of the disease being treated in a statistically significant manner, to slow the deterioration of a progressive disease in a statistically significant manner, or to prevent the onset of other related symptoms or diseases in a statistically significant manner, or any combination thereof. In some embodiments, an effective amount of a pharmaceutical composition or engineered extracellular vesicle is an amount sufficient to inhibit or treat a disease with minimal or no toxicity in a subject, excluding the presence of one or more adverse side effects. An effective amount or dose may be administrated once or multiple times over a given period of time. The effective amount or dosage may depend on the purpose of the treatment, and can be determined by one skilled in the art according to the needs of the subject. When referring to an individual active ingredient administrated alone, the effective amount or dosage refers to the amount of that ingredient alone. When referring to a combination, an effective amount or dosage refers to combined amounts of the active ingredients that produce a therapeutic effect, whether administrated serially or simultaneously.

### Engineered PSD Proteins

The present application provides an engineered phosphatidylserine decarboxylase (PSD protein), which comprises the following three functional domains: X₁X₂X₃X₄(X₅)ₘX₆(X₇)ₙX₈X₉X₁₀ at positions 114-139, X₁₁X₁₂X₁₃X₁₄X₁₅X₁₆X₁₇X₁₈X₁₉X₂₀ at positions 191-120, and X₂₁X₂₂X₂₃ at positions 307-309, referring to SEQ ID NO: 1;
wherein X₁ and X₂ are each independently Phe, Trp, Leu, Val, Ile or Tyr;
X₄ and X₆ are each independently Arg, Lys, Gln or Asn;
X₈, X₁₁, and X₂₀ are Pro;
X₁₀ is Asp, Glu or Asn;
X₁₄ is Tyr, Trp, Phe, Thr or Ser;
X₁₅, X₁₈, and X₁₉ are each independently His, Asn, Gln, Lys, or Arg;
X₂₁ is Gly;
X₂₂ is Ser;
X₂₃ is Ser, Thr or Val;
X₃, X₅, X₇, X₉, X₁₂, X₁₃, X₁₆, and X₁₇ are each independently any amino acid, m and n respectively represent m and n repeated amino acids, wherein m=1-6, 2-5, 3-4, or any positive integer within such a numerical range, and n=1-12, 2-11, 3-10, 4-9, 5-8, 6-7, or any positive integer within such a numerical range.

Particularly, the amino acid sequence of SEQ ID NO: 1 is:

MKKNGRDNNFYHLYKNKYLITGVTILSFILMFQYKYHEVLTLHDNSENA
VQSSKLFWARLLFGRTRSRITGQILKMEIPNTYRLFIFNFLIKYMHINK
EEIKYPIESYKSIGDFFSRYIREETRPIGDVSDYSIVSPCDSELIDYGE
LTSEYLENIKGVKFNVNTFLGSKFQKKHNDGSTKFFYAIFYLSPKKYHH
FHAPFNFKYKIRRHISGELFPVFQGMFKFINNLFNINERVILSGEWKGG
NVYYAAISAYNVGNIKIINDEELVTNNLRHQLSYMGGDINTKIFDSYKS
VEVGDEIGEFRMGSSIVVIFENKKDFSWNVNQNQTVSVGQRLGGIGEPV
KEENRFIKIRS

Particularly, the full-length cDNA sequence encoding the wild-type PSD protein is represented b SE ID NO: 2:

atgaagaagaatggaagagacaacaacttctaccacttgtacaagaaca
agtacctgatcacgggtgtgacaatcctgtccttcatcctcatgtttca
atacaagtaccatgaagtgctaaccctacacgataatagtgaaaatgct
gtacagagcagtaagcttttctgggcgcgactcctcttcggacgaacaa
ggagtcgaattacagggcaaatattaaaaatggaaatcccaaacacata
cagattgttcattttcaattttttaattaaatacatgcacatcaataag
gaagaaataaaatacccaatagagtcttacaaatccatcggagattttt
tctcccgttatattagagaggaaacgagacccattggagatgttagtga
ttactctatagtcagtccatgtgacagtgaactcatagattacggagaa
ttaacctcagaatatctagaaaatattaagggagtcaaatttaatgtaa
acactttcttgggatccaaattccagaagaagcataatgatggaagtac
caaatttttttatgccattttttatttaagtccaaaaaaataccaccat
tttcatgccccttttaatttcaagtacaaaattaggagacacatatctg
gagaattatttccagtttttcaaggcatgtttaaatttattaacaacct
ctttaatattaacgagagggtaatcctgtccggggaatggaaaggtggc
aatgtgtattatgccgccattagtgcttacaatgtaggaaatattaaaa
ttattaatgatgaagaattggttacgaataatttaaggcatcagttaag
ctacatgggaggagatatcaacaccaagattttcgactcctataaaagt
gtcgaagttggagacgaaattggggaattcagaatgggctcatccattg
ttgtaatttttgaaaataaaaaggacttctcctggaatgtcaaccaaaa
tcaaactgtttccgtaggccagaggcttggcgggatcggtgaacccgtc
aaggaggaaaacaggttcatcaaaattagaagctga

The amino acids herein may be natural amino acids or non-natural amino acids, represented by three letters or single letters known in the art, for example: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). When amino acids with similar structures and properties are replaced with each other, it often has little effect on the structure and properties of the protein, and can be used interchangeably.

In some embodiments, m may be, for example, 1, 2, 3, 4, 5 or 6, preferably m is 6. In some embodiments, n may be, for example, 1, 2, 3, 4, 5, 6, 7, 8, 9, 10, 11 or 12, preferably 12. In some embodiments, m is 6, and n is 12.

In some embodiments, the PSD protein comprises the following three functional domains: FFXRX₆RX₁₂PXD at positions 114-139, PXXYHXXHXP at positions 191-120, and GSS/GST at positions 307-309, referring to the wild-type PSD protein represented by SEQ ID NO: 1; wherein each X is independently any amino acid.

Since the PSD protein in most eukaryotes is expressed on the mitochondrial membrane and has low solubility, and pkPSD naturally exists in a part of secretable forms, in some embodiments, the engineered PSD protein is a PSD protein derived from Plasmodium knowlesi.

In some embodiments, the engineered PSD protein is a truncated PSD protein with a deletion of positions 1-45 from the N-terminus referring to the wild-type PSD protein represented by SEQ ID NO: 1, for example, it may be a truncated PSD protein with a deletion of positions 1-44, 1-43, 1-42, 1-41, 1-40, 1-39, 1-38, 1-37, 1-36, 1-35, 1-34 from the N-terminus referring to the wild-type PSD protein represented by SEQ ID NO: 1, preferably a truncated PSD protein with a deletion of positions 1-34 from the N-terminus referring to the wild-type PSD protein represented by SEQ ID NO: 1; and its amino acid sequence is represented by SEQ ID NO: 3:

MKYHEVLTLHDNSENAVQSSKLFWARLLFGRTRSRITGQILKMEIPNTY
RLFIFNFLIKYMHINKEEIKYPIESYKSIGDFFSRYIREETRPIGDVSD
YSIVSPCDSELIDYGELTSEYLENIKGVKFNVNTFLGSKFQKKHNDGST
KFFYAIFYLSPKKYHHFHAPFNFKYKIRRHISGELFPVFQGMFKFINNL
FNINERVILSGEWKGGNVYYAAISAYNVGNIKIINDEELVTNNLRHQLS
YMGGDINTKIFDSYKSVEVGDEIGEFRMGSSIVVIFENKKDFSWNVNQN
QTVSVGQRLGGIGEPVKEENRFIKIRS

In some embodiments, the engineered PSD protein comprises the amino acid sequence represented by SEQ ID NO: 3, or an amino acid sequence having at least 70%, 75%, 80%, 85%, 90%, 91%, 92%, 93%, 94%, 95%, 96%, 97%, 98%, or 99% similarity to the amino acid sequence represented by SEQ ID NO: 3.

In some embodiments, the engineered PSD protein comprises an insertion, deletion, or substitution mutation at one or two positions, wherein the positions are positions 217 and 219 referring to the wild-type PSD protein represented by SEQ ID NO: 1.

Amino acid substitutions, deletions and insertions may be accomplished by any well-known techniques, such as PCR-based techniques. The present application can also accomplish amino acid substitution by site-directed mutagenesis.

In some preferred embodiments, the engineered PSD protein comprises a substitution mutation at position 217 referring to the wild-type PSD protein represented by SEQ ID NO: 1, wherein the substitution mutation is P217H. In some more preferred embodiments, the engineered PSD protein comprises the amino acid sequence represented by SEQ ID NO: 4, or an amino acid sequence having at least 70%, 75%, 80%, 85%, 90%, 91%, 92%, 93%, 94%, 95%, 96%, 97%, 98% or 99% similarity to the amino acid sequence represented by SEQ ID NO: 4. In some embodiments, the amino acid sequence of the engineered PSD protein is represented by SEQ ID NO: 4.

Particularly, the amino acid sequence of SEQ ID NO: 4 is:

MKYHEVLTLHDNSENAVQSSKLFWARLLFGRTRSRITGQILKMEIPNTY
RLFIFNFLIKYMHINKEEIKYPIESYKSIGDFFSRYIREETRPIGDVSD
YSIVSPCDSELIDYGELTSEYLENIKGVKFNVNTFLGSKFQKKHNDGST
KFFYAIFYLSPKKYHHFHAPFNFKYKIRRHISGELFHVFQGMFKFINNL
FNINERVILSGEWKGGNVYYAAISAYNVGNIKIINDEELVTNNLRHQLS
YMGGDINTKIFDSYKSVEVGDEIGEFRMGSSIVVIFENKKDFSWNVNQN
QTVSVGQRLGGIGEPVKEENRFIKIRS
(the mutation site is underlined)

In some preferred embodiments, the engineered PSD protein comprises a substitution mutation at position 219 referring to the wild-type PSD protein represented by SEQ ID NO: 1, preferably, the substitution mutation is F219N. In some more preferred embodiments, the engineered PSD protein comprises the amino acid sequence represented by SEQ ID NO: 5, or an amino acid sequence having at least 70%, 75%, 80%, 85%, 90%, 91%, 92%, 93%, 94%, 95%, 96%, 97%, 98% or 99% similarity to the amino acid sequence represented by SEQ ID NO: 5. In some embodiments, the amino acid sequence of the engineered PSD protein is represented by SEQ ID NO: 5.

Particularly, the amino acid sequence of SEQ ID NO: 5 is:

MKYHEVLTLHDNSENAVQSSKLFWARLLFGRTRSRITGQILKMEIPNTY
RLFIFNFLIKYMHINKEEIKYPIESYKSIGDFFSRYIREETRPIGDVSD
YSIVSPCDSELIDYGELTSEYLENIKGVKFNVNTFLGSKFQKKHNDGST
KFFYAIFYLSPKKYHHFHAPFNFKYKIRRHISGELFPVNQGMFKFINNL
FNINERVILSGEWKGGNVYYAAISAYNVGNIKIINDEELVTNNLRHQLS
YMGGDINTKIFDSYKSVEVGDEIGEFRMGSSIVVIFENKKDFSWNVNQN
QTVSVGQRLGGIGEPVKEENRFIKIRS
(the mutation site is underlined)

In some embodiments, the engineered PSD protein is a PSD protein derived from humans, and the engineered PSD protein comprises the amino acid sequence represented by SEQ ID NO: 10, or an amino acid sequence having at least 70%, 75%, 80%, 85%, 90%, 91%, 92%, 93%, 94%, 95%, 96%, 97%, 98% or 99% similarity to the amino acid sequence represented by SEQ ID NO: 10.

Particularly, the amino acid sequence of SEQ ID NO: 10 is:

MATSVGHRCLGLLHGVAPWRSSLHPCEITALSQSLQPLRKLPFRAFRTD
ARKIHTAPARTMFLLRPLPILLVTGGGYAGYRQYEKYRERELEKLGLEI
PPKLAGHWEVALYKSVPTRLLSRAWGRLNQVELPHWLRRPVYSLYIWTF
GVNMKEAAVEDLHHYRNLSEFFRRKLKPQARPVCGLHSVISPSDGRILN
FGQVKNCEVEQVKGVTYSLESFLGPRMCTEDLPFPPAASCDSFKNQLVT
REGNELYHCVIYLAPGDYHCFHSPTDWTVSHRRHFPGSLMSVNPGMARW
IKELFCHNERVVLTGDWKHGFFSLTAVGATNVGSIRIYFDRDLHTNSPR
HSKGSYNDFSFVTHTNREGVPMRKGEHLGEFNLGSTIVLIFEAPKDFNF
QLKTGQKIRFGEALGSL

In some embodiments, the amino acid sequence of the engineered PSD protein is represented by SEQ ID NO: 13:

MLVTGGGYAGYRQYEKYRERELEKLGLEIPPKLAGHWEVALYKSVPTRL
LSRAWGRLNQVELPHWLRRPVYSLYIWTFGVNMKEAAVEDLHHYRNLSE
FFRRKLKPQARPVCGLHSVISPSDGRILNFGQVKNCEVEQVKGVTYSLE
SFLGPRMCTEDLPFPPAASCDSFKNQLVTREGNELYHCVIYLAPGDYHC
FHSPTDWTVSHRRHFPGSLMSVNPGMARWIKELFCHNERVVLTGDWKHG
FFSLTAVGATNVGSIRIYFDRDLHTNSPRHSKGSYNDFSFVTHTNREGV
PMRKGEHLGEFNLGSTIVLIFEAPKDFNFQLKTGQKIRFGEALGSL

In some embodiments, the engineered PSD protein is a PSD protein derived from a mouse, and the engineered PSD protein comprises the amino acid sequence represented by SEQ ID NO: 11, or an amino acid sequence having at least 70%, 75%, 80%, 85%, 90%, 91%, 92%, 93%, 94%, 95%, 96%, 97%, 98% or 99% similarity to the amino acid sequence represented by SEQ ID NO: 11.

Particularly, the amino acid sequence of SEQ ID NO: 11 is:

MAASGGRACVRSLRGGVLWRSSPCHYESTATRHFLGTLQKLPLQAGVRN
FHTAPVRSLFLLRPVPILLATGGGYAGYRQYEKYRERKLEKLGLEIPPK
LASHWEVSLYKSVPTRLLSRACGRLNQVELPYWLRRPVYSLYIWTFGVN
MTEAAVEDLHHYRNLSEFFRRKLKPQARPVCGLHCVTSPSDGKILTFGQ
VKNSEVEQVKGVTYSLESFLGPRANTEDLPFPPASSSDSFRNQLVTREG
NELYHCVIYLAPGDYHCFHSPTDWTISHRRHFPGSLMSVNPGMARWIKE
LFCHNERVVLTGDWKHGFFSLTAVGATNVGSIRIHFDRDLHTNSPRYSK
GSYNDLSFVTHANKEGIPMRKGEPLGEFNLGSTIVLIFEAPKDFNFRLK
AGQKIRFGEALGSL

In some embodiments, the amino acid sequence of the engineered PSD protein is represented by SEQ ID NO: 14:

MGLEIPPKLASHWEVSLYKSVPTRLLSRACGRLNQVELPYWLRRPVYSL
YIWTFGVNMTEAAVEDLHHYRNLSEFFRRKLKPQARPVCGLHCVTSPSD
GKILTFGQVKNSEVEQVKGVTYSLESFLGPRANTEDLPFPPASSSDSFR
NQLVTREGNELYHCVIYLAPGDYHCFHSPTDWTISHRRHFPGSLMSVNP
GMARWIKELFCHNERVVLTGDWKHGFFSLTAVGATNVGSIRIHFDRDLH
TNSPRYSKGSYNDLSFVTHANKEGIPMRKGEPLGEFNLGSTIVLIFEAP
KDFNFRLKAGQKIRFGEALGSL

In some embodiments, the engineered PSD protein is a PSD protein derived from Escherichia coli, and the engineered PSD protein comprises the amino acid sequence represented by SEQ ID NO: 12, or an amino acid sequence having at least 70%, 75%, 80%, 85%, 90%, 91%, 92%, 93%, 94%, 95%, 96%, 97%, 98% or 99% similarity to the amino acid sequence represented by SEQ ID NO: 12.

Particularly, the amino acid sequence of SEQ ID NO: 12 is:

MLNSFKLSLQYILPKLWLTRLAGWGASKRAGWLTKLVIDLFVKYYKVDM
KEAQKPDTASYRTFNEFFVRPLRDEVRPIDTDPNVLVMPADGVISQLGK
IEEDKILQAKGHNYSLEALLAGNYLMADLFRNGTFVTTYLSPRDYHRVH
MPCNGILREMIYVPGDLFSVNHLTAQNVPNLFARNERVICLFDTEFGPM
AQILVGATIVGSIETVWAGTITPPREGIIKRWTWPAGENDGSVALLKGQ
EMGRFKLGSTVINLFAPGKVNLVEQLESLSVTKIGQPLAVSTETFVTPD
AEPAPLPAEEIEAEHDASPLVDDKKDQV

### Fusion Protein

The present application further provides a fusion protein comprising the engineered PSD protein according to any one of the preceding items.

In some embodiments, the fusion protein further comprises a sequence promoting the solubility of the PSD protein, preferably a GST sequence. The fusion protein comprising the GST sequence according to the present application has high purification purity, mild purification conditions, unaffected protein activity and high solubility.

In some embodiments, the fusion protein further comprises one or two or three or more signal peptides of secretory proteins. In order to achieve autocrine secretion of engineered PSD protein in vivo, the present application needs to add a signal peptide to enable the cell to secrete the engineered PSD protein outside the cell to react with extracellular vesicles.

The present application does not limit the type of secretory protein, which may be any secretory protein, for example, it may be one or more selected from the group consisting of: antibody, cytokine, protein hormone and digestive enzyme. Cytokine is a class of small molecule proteins with a wide range of biological activities, and they are synthesized and secreted by immune cells (such as monocytes, macrophages, T cells, B cells, NK cells, etc.) and certain non-immune cells (endothelial cells, epidermal cells, fibroblasts, etc.) upon stimulation. Cytokines generally regulate cell growth, differentiation and effects by binding to corresponding receptors, thereby regulating immune responses. Cytokines (CKs) are low molecular weight soluble proteins produced by a variety of cells induced by immunogens, mitogens or other stimulants. They have multiple functions such as regulating innate immunity and adaptive immunity, hematopoiesis, cell growth, APSC multipotent cells and damaged tissue repair. The cytokines may be, for example, interleukin, interferon, tumor necrosis factor superfamily, colony stimulating factor, chemokine, growth factor, and the like. Protein hormones include, for example, insulin, glucagon, growth hormone, thyroid hormone, thyroid stimulating hormone, etc. Digestive enzymes include, for example, amylase, pepsin, trypsin, cellulase, lipase, and the like.

In some embodiments, interleukins may be, for example, IL-2, IL-7, IL-10, IL-11, IL-12, IL-15, IL-18, IL-21, IL-23, etc.; members of the tumor necrosis factor family may be, for example, TNF, LTA, LTB, FASLG, TNFSF8, TNFSF9, TNFSF10, TNFSF11, TNFSF12, TNFSF13, TNFSF14, TNFSF15, TNFSF18, EDA, etc.; interferons may be, for example, INF-α, INF-β, INF-γ, etc.

In some embodiments, the fusion protein comprises an amino acid sequence selected from any one of SEQ ID NOs: 6-8, or an amino acid sequence having at least 70%, 75%, 80%, 85%, 90%, 91%, 92%, 93%, 94%, 95%, 96%, 97%, 98% or 99% similarity to the amino acid sequence selected from any one of SEQ ID NOs: 6-8.

Particularly, the amino acid sequence of SEQ ID NO: 6 is:

MCHQQLVISWFSLVFLASPLVAMSPILGYWKIKGLVQPTRLLLEYLEEK
YEEHLYERDEGDKWRNKKFELGLEFPNLPYYIDGDVKLTQSMAIIRYIA
DKHNMLGGCPKERAEISMLEGAVLDIRYGVSRIAYSKDFETLKVDFLSK
LPEMLKMFEDRLCHKTYLNGDHVTHPDFMLYDALDVVLYMDPMCLDAFP
KLVCFKKRIEAIPQIDKYLKSSKYIAWPLQGWQATFGGGDHPPKSDGST
SGSGHHHHHHSAGLVPRGSGSGSGSDYKDDDDKGGSSMKYHEVLTLHDN
SENAVQSSKLFWARLLFGRTRSRITGQILKMEIPNTYRLFIFNFLIKYM
HINKEEIKYPIESYKSIGDFFSRYIREETRPIGDVSDYSIVSPCDSELI
DYGELTSEYLENIKGVKFNVNTFLGSKFQKKHNDGSTKFFYAIFYLSPK
KYHHFHAPFNFKYKIRRHISGELFPVFQGMFKFINNLFNINERVILSGE
WKGGNVYYAAISAYNVGNIKIINDEELVTNNLRHQLSYMGGDINTKIFD
SYKSVEVGDEIGEFRMGSSIVVIFENKKDFSWNVNQNQTVSVGQRLGGI
GEPVKEENRFIKIRS

The amino acid sequence of SEQ ID NO: 7 is:

MCHQQLVISWFSLVFLASPLVAMSPILGYWKIKGLVQPTRLLLEYLEEK
YEEHLYERDEGDKWRNKKFELGLEFPNLPYYIDGDVKLTQSMAIIRYIA
DKHNMLGGCPKERAEISMLEGAVLDIRYGVSRIAYSKDFETLKVDFLSK
LPEMLKMFEDRLCHKTYLNGDHVTHPDFMLYDALDVVLYMDPMCLDAFP
KLVCFKKRIEAIPQIDKYLKSSKYIAWPLQGWQATFGGGDHPPKSDGST
SGSGHHHHHHSAGLVPRGSGSGSGSDYKDDDDKGGSSMKYHEVLTLHDN
SENAVQSSKLFWARLLFGRTRSRITGQILKMEIPNTYRLFIFNFLIKYM
HINKEEIKYPIESYKSIGDFFSRYIREETRPIGDVSDYSIVSPCDSELI
DYGELTSEYLENIKGVKFNVNTFLGSKFQKKHNDGSTKFFYAIFYLSPK
KYHHFHAPFNFKYKIRRHISGELFHVFQGMFKFINNLFNINERVILSGE
WKGGNVYYAAISAYNVGNIKIINDEELVTNNLRHQLSYMGGDINTKIFD
SYKSVEVGDEIGEFRMGSSIVVIFENKKDFSWNVNQNQTVSVGQRLGGI
GEPVKEENRFIKIRS
(the mutation site is underlined)

The amino acid sequence of SEQ ID NO: 8 is:

MCHQQLVISWFSLVFLASPLVAMSPILGYWKIKGLVQPTRLLLEYLEEK
YEEHLYERDEGDKWRNKKFELGLEFPNLPYYIDGDVKLTQSMAIIRYIA
DKHNMLGGCPKERAEISMLEGAVLDIRYGVSRIAYSKDFETLKVDFLSK
LPEMLKMFEDRLCHKTYLNGDHVTHPDFMLYDALDVVLYMDPMCLDAFP
KLVCFKKRIEAIPQIDKYLKSSKYIAWPLQGWQATFGGGDHPPKSDGST
SGSGHHHHHHSAGLVPRGSGSGSGSDYKDDDDKGGSSMKYHEVLTLHDN
SENAVQSSKLFWARLLFGRTRSRITGQILKMEIPNTYRLFIFNFLIKYM
HINKEEIKYPIESYKSIGDFFSRYIREETRPIGDVSDYSIVSPCDSELI
DYGELTSEYLENIKGVKFNVNTFLGSKFQKKHNDGSTKFFYAIFYLSPK
KYHHFHAPFNFKYKIRRHISGELFPVNQGMFKFINNLFNINERVILSGE
WKGGNVYYAAISAYNVGNIKIINDEELVTNNLRHQLSYMGGDINTKIFD
SYKSVEVGDEIGEFRMGSSIVVIFENKKDFSWNVNQNQTVSVGQRLGGI
GEPVKEENRFIKIRS
(the mutation site is underlined)

### Nucleic Acid Molecules, Expression Vectors and Engineered Cells

The present application further provides a nucleic acid molecule encoding the engineered PSD protein according to any one of the preceding items, or the fusion protein according to any one of the preceding items.

In some embodiments, the sequence of the nucleic acid molecule is represented by SEQ ID NO: 9, or is a nucleotide sequence having at least 70%, 75%, 80%, 85%, 90%, 91%, 92%, 93%, 94%, 95%, 96%, 97%, 98% or 99% similarity to SEQ ID NO: 9.

Particularly, the nucleotide sequence of SEQ ID NO: 9 is a codon-optimized cDNA sequence encoding a truncated PSD protein with deletions of positions 1 to 34 from N-terminus referring to the wild-type PSD protein represented by SEQ ID NO: 1; and the nucleotide sequence of SEQ ID NO: 9 is:

ATGAAATACCACGAAGTCCTCACCCTCCATGACAACTCAGAAAACGCCG
TCCAGTCTTCAAAACTGTTCTGGGCTAGGCTGTTGTTCGGCCGCACGAG
AAGTCGGATCACTGGCCAGATATTGAAAATGGAAATACCGAATACCTAT
AGGTTGTTTATTTTCAATTTCCTGATCAAATACATGCACATCAATAAGG
AGGAGATCAAATACCCTATCGAATCATATAAAAGCATCGGCGACTTTTT
TTCTAGGTACATTAGAGAAGAAACACGGCCAATTGGTGACGTCAGCGAT
TACAGTATCGTGAGCCCTTGCGATAGCGAGTTGATTGACTATGGTGAAC
TCACAAGCGAATACCTCGAAAATATCAAAGGCGTGAAATTCAACGTTAA
CACCTTTCTGGGGTCAAAATTTCAAAAGAAGCACAACGATGGTAGCACG
AAATTCTTCTATGCTATCTTCTACTTGTCCCCCAAGAAGTATCACCATT
TCCATGCTCCATTTAACTTCAAATATAAGATCCGGAGACACATATCTGG
CGAACTCTTCCCCGTTTTCCAAGGCATGTTTAAGTTTATCAATAACCTG
TTCAATATTAACGAGAGAGTGATACTTAGTGGGGAGTGGAAGGGGGGTA
ATGTTTATTATGCTGCTATTAGCGCCTACAATGTTGGGAACATTAAGAT
TATCAATGACGAAGAGCTGGTCACCAACAATCTGAGACACCAGCTGTCT
TATATGGGCGGGGACATTAACACAAAGATCTTTGATTCTTATAAGAGCG
TTGAAGTAGGGGACGAGATTGGCGAGTTCAGGATGGGCTCTTCAATTGT
GGTAATCTTCGAAAACAAAAAGGACTTTTCCTGGAATGTTAACCAGAAT
CAAACTGTGAGTGTAGGACAAAGATTGGGAGGGATCGGGGAGCCTGTGA
AGGAAGAGAACCGCTTCATTAAGATCCGCTCCTAG

The present application further provides an expression vector comprising the nucleic acid molecule according to any one of the preceding items.

In some embodiments, the expression vector can be any type of plasmid, and it refers to a circular double-stranded DNA loop into which additional DNA fragments can be inserted, for example, by standard molecular cloning techniques, wherein the plasmid may be pET-42a, PX458, or pcDNA3.1, etc.

The present application further provides an engineered cell comprising any one of the above nucleic acid molecules and a protein encoding a cargo.

In some embodiments, the cargo protein is one or two or three or more selected from the group consisting of: a therapeutic peptide, a DNA binding protein, an RNA binding protein, a fluorescent protein, an enzyme, or a linker for connecting a therapeutic compound.

In some embodiments, the therapeutic peptide can be a member of the human interleukin family (e.g., IL-2, IL-7, IL-10, IL-11, IL-12, IL-15, IL-18, IL-21, and IL-23), a member of the tumor necrosis factor family (e.g., TNF, LTA, LTB, FASLG, TNFSF8, TNFSF9, TNFSF10, TNFSF11, TNFSF12, TNFSF13, TNFSF14, TNFSF15, TNFSF18, and EDA), an interferon (IN F-α, INF-β and INF-γ), a T cell engager (e.g., 4-1BB, OX40, CD28, CD40, CD40L, CD47, CD27, CD70, CD80, CD86, GITRL, ICOSL, CD155, CD112, TIM-3, and BTLA), and other cytokines (e.g., G-CSF, EPO, TPO, GM-CSF, EGF, bFGF, FVIIa, ATIII, TNK, α-Glucosidase, BMP-2, and hirudin).

### Method for Preparing Engineered Extracellular Vesicles and Engineered Extracellular Vesicles

The present application provides a method for preparing an engineered extracellular vesicle, which comprises the following steps:

culturing the engineered cell according to any one of the preceding items, and isolating the extracellular vesicle secreted by the engineered cell from a culture medium.

In some embodiments, the culture conditions are those known in the art, including appropriate culture medium, temperature, concentration of carbon dioxide, and the like. The type of engineered cells is not particularly limited, and may be animal cells (e.g., monkey cells, mouse cells, etc.) or human cells, examples of which include but are not limited to: HEK293F cells, HEK293T cells, Vero cells, CHO cells, HeLa cells, HuH7 cells, HEK-293 cells, and macrophages, etc.

In some embodiments, the culture medium is preferably culture supernatant. In some embodiments, the separation is sometimes also referred to as extraction or enrichment, and examples of which include but are not limited to: differential centrifugation, density gradient centrifugation, ultrafiltration centrifugation, magnetic bead immunoassay, etc. The separation can also be performed by using a known kit.

The present application further provides an engineered extracellular vesicle, which comprises the fusion protein according to any one of the preceding items and a cargo protein.

In some embodiments, the cargo protein is a cargo protein according to any one of the preceding items.

Pharmaceutical Composition, Pharmaceutical Use, and Method for Diagnosing, Treating and/or Preventing Diseases

The present application provides a pharmaceutical composition comprising the engineered extracellular vesicle according to any one of the preceding items or the engineered extracellular vesicle prepared by the method according to any one of the preceding items, and a pharmaceutically acceptable carrier.

In some embodiments, the pharmaceutically acceptable carrier can be selected from the group consisting of: water, buffered aqueous solution, isotonic saline solution such as PBS (phosphate buffered saline), glucose, mannitol, dextrose, lactose, starch, magnesium stearate, cellulose, magnesium carbonate, 0.3% glycerol, hyaluronic acid, ethanol or polyalkylene glycol such as polypropylene glycol, triglycerides, etc.

In some embodiments, the pharmaceutical composition according to the present application may further comprise lubricants, preservatives, stabilizers, wetting agents, emulsifiers, salts affecting osmotic pressure, buffers, coloring substances, flavoring substances, and/or aromatic substances as additives.

The pharmaceutical composition according to the present application may be in any suitable dosage form. For example, injection, suspension, and emulsion, etc. The pharmaceutical composition according to the present application can be administrated into the body by known means. For example, delivery to the tissue of interest is by intramuscular injection, optionally administrated via intravenous, transdermal, intranasal, oral, mucosal, or other delivery methods. Such administration may be carried out via a single dose or multiple doses. It is understood by those skilled in the art that, the actual dosage to be administrated herein may vary greatly depending on a variety of factors, such as the target cell, organism type or tissue thereof, the general condition of the subject to be treated, the route of administration, the mode of administration, and the like.

The present application further provides use of the fusion protein according to any one of the preceding items, the engineered extracellular vesicle according to any one of the preceding items, or the engineered extracellular vesicle prepared by the method according to any one of the preceding items in the preparation of a medicament for diagnosing, treating and/or preventing diseases.

The present application further provides a method for diagnosing, treating and/or preventing a disease, comprising administrating to a subject in need thereof an effective amount of the engineered extracellular vesicle according to any one of the preceding items, or the engineered extracellular vesicle prepared by the method according to any one of the preceding items.

The engineered extracellular vesicle and pharmaceutical composition according to the present application can be used to treat a variety of diseases, such as immune diseases or cancer. The engineered extracellular vesicle and pharmaceutical composition according to the present application can be used to prevent a disease in a subject or for other therapeutic applications to a subject in need thereof.

The engineered extracellular vesicle and pharmaceutical composition according to the present application can be administrated to a variety of different subjects, including but not limited to: mammal, human, non-human mammal, domesticated animal (e.g., laboratory animal, household pet or livestock), non-domesticated animal (e.g., wild animal), dog, cat, rodent, mouse, hamster, cow, bird, chicken, fish, pig, horse, goat, sheep, rabbit, and any combination thereof. In some embodiments, the subject is a human.

The engineered extracellular vesicle and pharmaceutical composition according to the present application can be used as a therapeutic agent, for example, a therapeutic product that can be administrated to subjects in need. The therapeutic effects of the engineered extracellular vesicle and pharmaceutical composition according to the present application can be obtained in a subject by reducing, inhibiting, alleviating or eradicating a disease state (including but not limited to its symptoms).

Administration of an effective amount or dose of the engineered extracellular vesicle or pharmaceutical composition according to the present application to a subject can be performed via one or more routes, and can occur once or multiple times within a given period of time. A person of ordinary skill in the art will understand that, the amount, duration, and frequency of administration of the engineered extracellular vesicle and pharmaceutical composition thereof according to the present application to a subject in need thereof depends on several factors, including, for example, the health condition of the subject, the particular disease or condition of the subject, the grade or level of the particular disease or condition of the subject, additional treatments that the subject is receiving or has received, etc. Exemplary routes of administration include systemic, cutaneous, subcutaneous, intravenous, intraarterial, subdural, intramuscular, intracranial, intrasternal, intratumoral, and intraperitoneal. In addition, the engineered extracellular vesicle and pharmaceutical composition according to the present application can be administrated to a subject via other routes of administration, such as by inhalation, or oral, dermal, intranasal or intrathecal administration.

### EXAMPLES

### Example 1: Construction of Engineered Cells

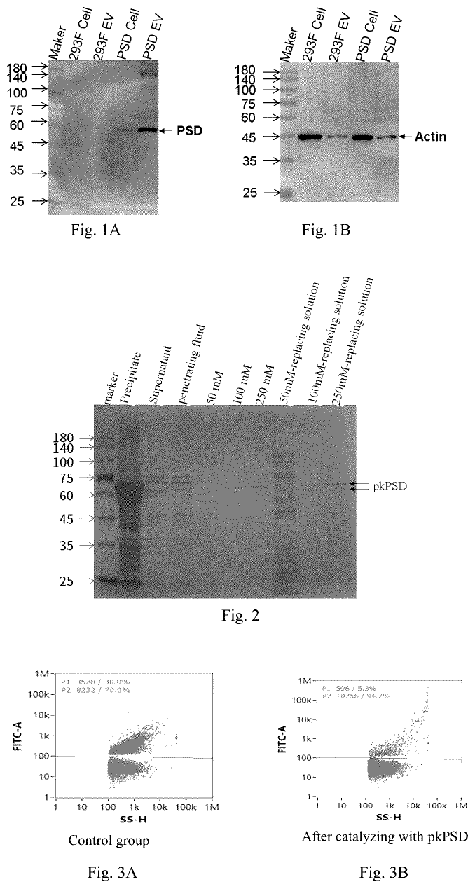

The engineered cells were constructed by the following method, wherein the pkPSD protein was secreted into the cell culture supernatant, and the pkPSD protein catalyzed the cleavage of phosphatidylserine carboxyl group during the cell secretion of extracellular vesicles. The particular steps are as follows:
(1) Plasmid Construction: the pkPSD expression plasmid was designed to express the fusion protein hIL12BSp-GST-his-Flag-pkPSD_35_319 (the amino acid sequence is represented by SEQ ID NO: 6);
(2) EV Preparation: The plasmid was transferred into HEK293 (HEK293F) to construct a mixed clone cell, named PSD cell, which was used as the experimental group. The control group was HEK293F cells without plasmid transfer. The cells of the experimental group and the control group were cultured, and the cell supernatant was harvested to extract EVs. At the same time, the cell lysate was prepared for later use; particularly, the extracellular vesicles extracted from the PSD cells were PSD EVs (hereinafter, PSD EVs all refer to the pkPSD EVs prepared in Example 1);
(3) Western Blot Analysis: The cell precipitations, supernatants, and extracted extracellular vesicles of the experimental group cells and the control group cells were loaded according to the same amount of protein for Western blot analysis, and the expression of pkPSD in each sample was analyzed. The analysis results are shown in FIGS. 1A and 1B.
(4) Nanoflow Cytometry Assay: Nanoflow cytometer was used to detect the number of particles and analyze the particle size of the extracellular vesicles in the experimental group and the control group.

As can be seen from FIG. 1A, after PSD was transiently transferred into HEK293F cells, the Flag tag fused with PSD was detected. There was no target protein in the HEK293F cells and EVs of the control group, while the expression of Flag fusion protein could be detected in the PSD cells and EVs of the experimental group. The Actin protein in FIG. 1B is a housekeeping gene in the detected cells and EVs, and reflecting whether the loading amounts is consistent.

### Example 2: pkPSD can Reduce the Content of Phosphatidylserine on the Surface of Extracellular Vesicles

Plasmid Construction and Protein Purification: pET-42a was used as the plasmid backbone to express his-GST-pkPSD_35_319 protein, its amino acid sequence is represented by SEQ ID NO: 15 (MSPILGYWKIKGLVQPTRLLLEYLEEKYEEHLYERDEGDKWRNKKFELGLEFPNLPYYI DGDVKLTQSMAIIRYIADKHNMLGGCPKERAEISMLEGAVLDIRYGVSRIAYSKDFETLK VDFLSKLPEMLKMFEDRLCHKTYLNGDHVTHPDFMLYDALDVVLYMDPMCLDAFPKL VCFKKRIEAIPQIDKYLKSSKYIAWPLQGWQATFGGGDHPPKSDGSTSGSGHHHHHHSA GLVPRGSTAIGMKETAAAKFERQHMDSPDLGTGGGSGMKYHEVLTLHDNSENAVQSSK LFWARLLFGRTRSRITGQILKMEIPNTYRLFIFNFLIKYMHINKEEIKYPIESYKSIGDFFSR YIREETRPIGDVSDYSIVSPCDSELIDYGELTSEYLENIKGVKFNVNTFLGSKFQKKHNDG STKFFYAIFYLSPKKYHHFHAPFNFKYKIRRHISGELFPVFQGMFKFINNLFNINERVILSG EWKGGNVYYAAISAYNVGNIKIINDEELVTNNLRHQLSYMGGDINTKIFDSYKSVEVGD EIGEFRMGSSIVVIFENKKDFSWNVNQNQTVSVGQRLGGIGEPVKEENRFIKIRS). The expression plasmid was transferred into Escherichia coli to obtain the expression strain. The strain was cultured until OD600 was about 1.0, and 0.5 mM IPTC was added to induce at 37° C. for 3 h. The expression strain cells were collected and ultrasonically disrupted, and the supernatant containing the recombinant fusion protein was collected by centrifugation. The his-GST-pkPSD_35_319 protein was purified by his-affinity chromatography. The results for electrophoresis identification are shown in FIG. 2. As can be seen from FIG. 2, a large amount of protein was expressed in the precipitate, and soluble recombinant protein was present in the supernatant. A large amount of non-target proteins were eluted after elution with a low concentration of 50 mM imidazole. After elution with 100 mM and 250 mM imidazole, pure pkPSD protein (69.96 kD) and its mature protein (62 kD) were obtained.
(1) After pkPSD catalysis, the PS content on the surface of extracellular vesicles was decreased.

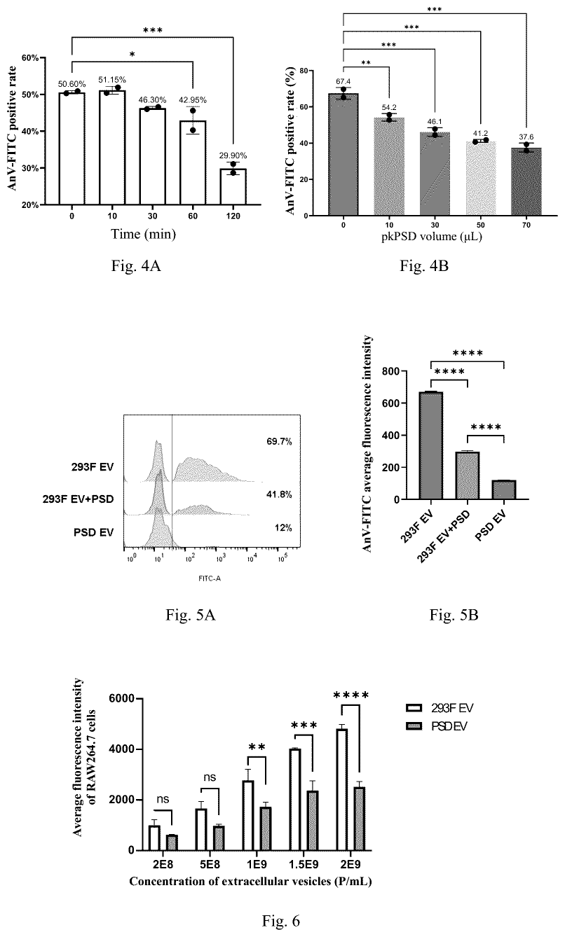

After the extracellular vesicles were incubated with pkPSD protein or pkPSD protein dialysate at 37° C. for 3 h, the phosphatidylserine on the surface of the extracellular vesicles was labeled with AnV-FITC, the unbound free dye was removed by SEC (size exclusion chromatography), and the particle number detection and positive rate analysis were performed by nano-flow cytometer. The results are shown in FIG. 3B, and FIG. 3A is the analysis results of the control group. By comparing FIGS. 3A and 3B, it can be seen that after catalysis by addition of pkPSD, the AnV-FITC positive rate on the surface of the extracellular vesicles was decreased, indicating that the phosphatidylserine content on the surface of the extracellular vesicles was reduced.
(2) The pkPSD enzyme activity increased with the increase of incubation time and enzyme amount.

The extracellular vesicles were incubated with pkPSD protein at 37° C. for different time periods (0 min, 10 min, 30 min, 60 min, or 120 min) or incubated with different amounts of pkPSD protein (0 μL, 10 μL, 30 μL, 50 μL, or 70 μL) at 37° C. for 3 h. Phosphatidylserine on the surface of extracellular vesicles was labeled with AnV-FITC, unbound free dye was removed by SEC (size exclusion chromatography), and the particle number detection and positive rate analysis were performed by nano-flow cytometer. The results are shown in FIGS. 4A and 4B.

It can be seen from FIG. 4A that, as the reaction time increased, the AnV-FITC positive rate of extracellular vesicles was decreased, indicating that phosphatidylserine on the surface of extracellular vesicles was decreased. As can be seen from FIG. 4B, the phosphatidylserine on the surface of extracellular vesicles was decreased as the addition amount of pkPSD increased. This indicates that pkPSD has the activity of reducing phosphatidylserine on the surface of extracellular vesicles.

### Example 3: Extracellular Vesicles Extracted from Engineered Cells have the Advantage of Low Natural PS Content

Referring to the AnV-FITC staining method of Example 2, AnV-FITC staining was performed on the three extracellular vesicles, namely, extracellular vesicles 293F EV extracted from HEK293F, extracellular vesicles 293F EV+PSD obtained by adding pkPSD in vitro to catalyze 293F EV prepared in Example 2, and extracellular vesicles PSD EV extracted from PSD-engineered cells in Example 1. The FITC positivity rate of extracellular vesicles was detected by nano-flow cytometry detection technology. The raw data were imported into FlowJo analysis software for analysis. The results are shown in FIGS. 5A and 5B. 293F EV+PSD obtained by catalyzing 293F EV in vitro with PSD can significantly reduce the AnV-FITC intensity relative to 293F EV, while PSD EV (self-expressing PSD) can further reduce the AnV-FITC intensity. This indicates that PSD-engineered cells can obtain extracellular vesicles with low PS content during the extracellular vesicle purification process, eliminating the need of catalysis through in vitro addition of PSD, and the enzyme activity is more advantageous than that obtained by in vitro catalysis (longer processing time).

### Example 4: Extracellular Vesicles Based on Engineered PSD can Significantly Reduce Macrophage Phagocytosis In Vitro

The 293F EV and PSD EV of Example 1 were stained with cell membrane fluorescent dyes, and different concentrations of extracellular vesicles of the experimental group and the control group were added to incubate with RAW264.7 macrophages at 37° C. for 2 h. The free extracellular vesicles were washed three times with PBS, and the cells were resuspended to subject to flow cytometry detection. The test results are shown in FIG. 6, wherein the ordinate represents the average fluorescence intensity of macrophages, reflecting the number of fluorescently labeled extracellular vesicles phagocytosed in the cells. As can be seen from FIG. 6, PSD EV can significantly reduce the average fluorescence intensity of RAW264.7 cells, indicating that PSD EV can significantly reduce macrophage phagocytosis in vitro.

### Example 5: Extracellular Vesicles Based on Engineered PSD can Prolong the Circulation Time in Mice

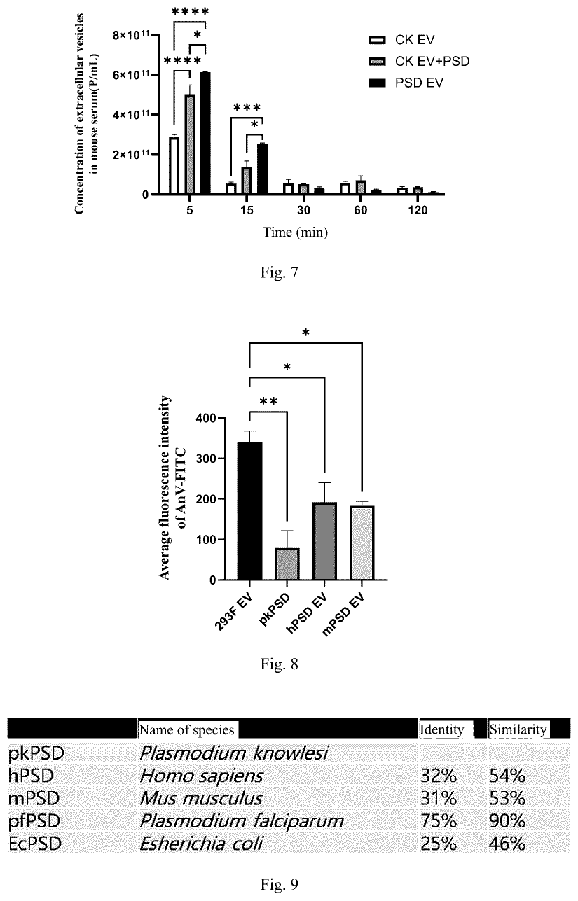

The same number of particles of 293F EVs (CK EVs), EVs (CK EVs+PSD) obtained by adding pkPSD prepared in Example 2 to catalyze 293F EV in vitro, and PSD EVs were injected into mice by tail vein, and the extracellular vesicle content in mouse serum was detected at different time points. The results are shown in FIG. 7.

In FIG. 7, CK EV is 293F EV without any treatment, CK EV+PSD is a sample obtained by adding pkPSD prepared in Example 2 to 293F EV for in vitro catalysis, and PSD EV is a sample of autocrine PSD. It can be seen from FIG. 7 that, compared with the CK EV group, the CK EV+PSD group and the PSD EV group had increased extracellular vesicles in the serum, indicating that the clearance of these two groups of extracellular vesicles in mice was reduced; at the same time, compared with the CK EV+PSD group, the number of residual extracellular vesicles in the serum of the PSD EV group was further increased, indicating that autocrine PSD has advantages over PSD obtained by in vitro catalysis and can prolong the circulation time of extracellular vesicles.

### Example 6: Mutations in the Conserved Sequences of PSD Family Proteins Affect their Enzyme Activity

PSD from Plasmodium knowlesi, humans (Homo sapiens), and mice (Mus musculus) (labeled as pkPSD (hIL12BSp-GST-his-Flag-pkPSD_35_319), hPSD (hIL12BSp-GST-his-Flag-hPSD_71_409), and mPSD (hIL12BSp-GST-his-Flag-mPSD_92_406), respectively) were transiently transfected into 293F cells, extracellular vesicles were extracted, and PS on the surface of extracellular vesicles was labeled with AnV-FITC. The AnV-FITC positivity rate/MFI was detected. The results are shown in FIG. 8. Compared with 293F EVs, the PS on the surface of EVs expressing PSD was significantly reduced.

The identity (indicating the proportion of identical amino acids in the amino acid sequences) and similarity (indicating the proportion of similar amino acids in the amino acid sequences) of the PSD of each species are shown in FIG. 9.

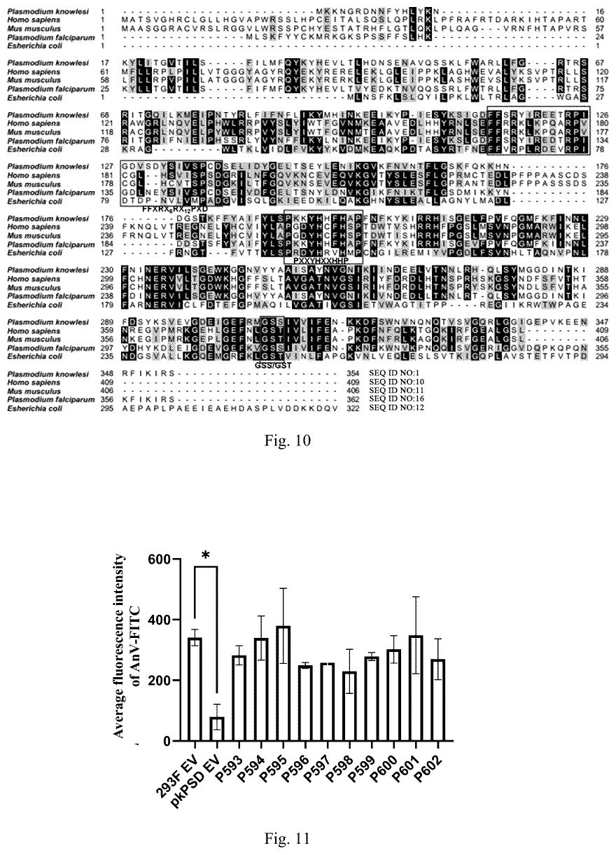

By performing multiple sequence alignment on PSD proteins of all species, it was found that PSD proteins comprise three conserved motifs: FFXRX₆RX₁₂PXD, PXXYHXXHXP, and GSS/GST, wherein X is any amino acid. Some of the representative species are listed in FIG. 10.

Furthermore, by performing point mutation on the above conserved sequences, it was confirmed that the above three motifs are key conserved motifs. The particular mutation sites are shown in Table 1, and the mutations are mutations made relative to the fusion protein hIL12BSp-GST-his-Flag-pkPSD_35_319.

TABLE 1

| Plasmid No. | Conserved motif | Mutation site indication | Specific mutation site |
|---|---|---|---|
| P593 | FFXRX₆RX₁₂PXD | FFXRX₆RX₁₂PXA | D139A |
| P594 | | AAXAX₆AX₁₂AXD | F114A, F115A, R117A, |
| | | | R124A, P137A |
| P595 | | AAXAX₆AX₁₂AXA | F114A, F115A, R117A, |
| | | | R124A, P137A, D139A |
| P596 | PXXYHXXHHP | PXXYAXXHHP | H195A |
| P597 | | PXXYHXXAHP | H198A |
| P598 | | PXXYAXXAHP | H195A, H198A |
| P599 | GSS/GST | ASS | G307A |
| P600 | | GTS | S308T |
| P601 | | GAS | S308A |
| P602 | | GSA | S309A |

Referring to the method of Example 1, the above mutant plasmid was constructed and transiently transfected into 293F cells, then the extracellular vesicles were extracted, and then AnV-FITC positive rate was detected. The results are shown in FIG. 11. The PS content on the surface of extracellular vesicles of pkPSD EV was significantly reduced as compared with that of 293F EV, and there was no significant difference relative to 293F EV after the key site mutations (P593˜P602), indicating the importance of the key motif to the enzyme activity of PSD.

### Example 7: pkPSD Mutation Site can Increase Enzyme Activity

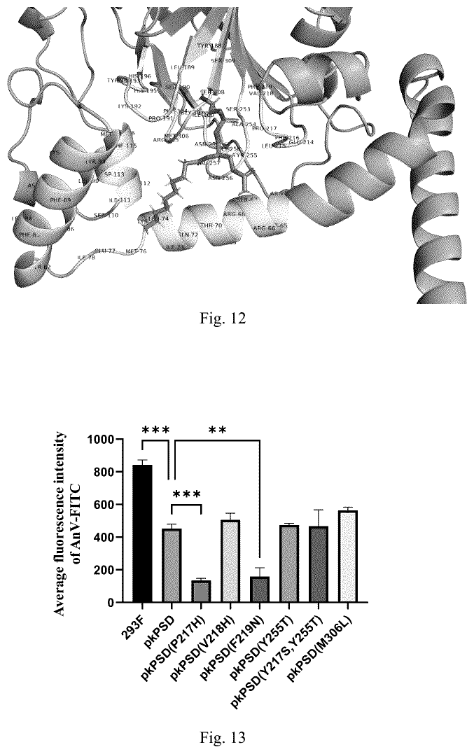

By simulating the substrate pocket of pkPSD obtained in Example 1, a schematic diagram of the three-dimensional structure of the protein pocket for the interaction between pkPSD and PS was obtained, as shown in FIG. 12. The particular amino acid ranges near the substrate pocket are: positions 64-78, 82-94, 110-116, 189-196, 214-219, 253-260, and 304-309 in the pkPSD structure. Based on the particular amino acid ranges near the substrate pocket, multiple mutation sites were designed: positions 217, 218, 219, 255 and 306. The mutant plasmids were constructed and transiently transfected into 293F cells, and then the extracellular vesicles were extracted for AnV-FITC detection. The test results are shown in FIG. 13. The PS content on the surface of extracellular vesicles of pkPSD wild-type EVs was reduced as compared with 293F EVs. pkPSD(P217H) EV and pkPSD(F219N) EV can further reduce the PS on the surface of extracellular vesicles as compared with wild-type pkPSD EVs.

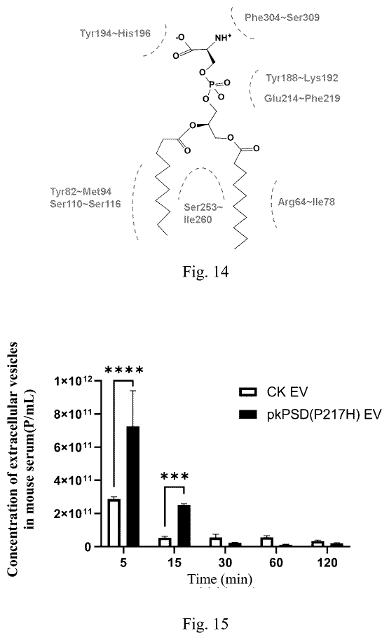

The structural planar schematic diagram of PS (10:0/10:0) and pkPSD protein obtained by simulating the substrate pocket of pkPSD prepared in Example 1 is shown in FIG. 14, particularly 10:0 and 10:0 respectively represent the ratios of saturated bonds and unsaturated bonds of the two side chains in the structure of phosphatidylserine PS, wherein the protein residues around PS are gray, PS is black, and the dotted line indicates that there may be interaction between PS and pkPSD. Theoretically, mutations in the amino acids in FIG. 14 may change the ability to bind to PS and further change the enzymatic activity of the protein.

### Example 8: pkPSD Mutant Still has the Function of Increasing the Circulation Time in Mice

The same number of particles of 293F EV (CK EV) and pkPSD (P217H) EV were injected into mice by tail vein, and the extracellular vesicle content in the mouse serum was detected at different time points. The results are shown in FIG. 15. Compared with CK EV, pkPSD(P217H) EV had an increased level of residual extracellular vesicles in mouse serum after tail vein injection, indicating that its clearance in the mouse body was reduced, and its circulation time was increased. This indicates that pkPSD mutant still has the function of prolonging the circulation time of extracellular vesicles.

The above is only description of preferred embodiments of the present application, and does not constitute any other form of limitation to the present application. Any technician familiar with the present profession may use the technical contents disclosed above to change or modify them into equivalent embodiments with equivalent changes. However, any simple modifications, equivalent changes and modifications made to the above embodiments based on the technical essence of the present application without departing from the content of the technical solutions of the present application still fall within the protection scope of the technical solutions of the present application.

1. An engineered phosphatidylserine decarboxylase PSD protein, comprising the following three functional domains: X₁X₂X₃X₄(X₅)ₘX₆(X₇)ₙX₈X₉X₁₀ at positions 114-139, X₁₁X₁₂X₁₃X₁₄X₁₅X₁₆X₁₇X₁₈X₁₉X₂₀ at positions 191-120, and X₂₁X₂₂X₂₃ at positions 307-309, referring to the wild-type PSD protein represented by SEQ ID NO: 1;
wherein X₁ and X₂ are each independently Phe, Trp, Leu, Val, Ile or Tyr;
X₄ and X₆ are each independently Arg, Lys, Gln or Asn;
X₈, X₁₁, and X₂₀ are Pro;
X₁₀ is Asp, Glu or Asn;
X₁₄ is Tyr, Trp, Phe, Thr or Ser;
X₁₅, X₁₈, and X₁₉ are each independently His, Asn, Gln, Lys, or Arg;
X₂₁ is Gly;
X₂₂ is Ser;
X₂₃ is Ser, Thr or Val;
X₃, X₅, X₇, X₉, X₁₂, X₁₃, X₁₆, and X₁₇ are each independently any amino acid, m and n respectively represent m and n repeated amino acids, wherein m=1-6, 2-5, 3-4, or any positive integer within such a numerical range, and n=1-12, 2-11, 3-10, 4-9, 5-8, 6-7, or any positive integer within such a numerical range.

2. The engineered PSD protein according to claim 1, comprising the following three functional domains: FFXRX₆RX₁₂PXD at positions 114-139, PXXYHXXHXP at positions 191-120, and GSS/GST at positions 307-309, referring to the wild-type PSD protein represented by SEQ ID NO: 1, wherein each X is independently any amino acid.

3. The engineered PSD protein according to claim 1, which is a PSD protein derived from Plasmodium knowlesi.

4. The engineered PSD protein according to claim 1, which is a truncated PSD protein with deletion of amino acids at positions 1-45 from the N-terminus.

5. The engineered PSD protein according to claim 1, which is a truncated PSD protein with deletion of amino acids at positions 1-34 from the N-terminus.

6. The engineered PSD protein according to claim 1, comprising an insertion, deletion or substitution mutation at one or two positions, wherein the positions are positions 217 and 219 referring to the wild-type PSD protein represented by SEQ ID NO: 1.

7. The engineered PSD protein according to claim 6, wherein the mutation is a substitution mutation.

8. The engineered PSD protein according to claim 7, wherein the substitution mutations are P217H and F219N.

9. The engineered PSD protein according to claim 1, comprising an amino acid sequence selected from any one of SEQ ID NOs: 3-5 and SEQ ID NOs: 10-12, or an amino acid sequence having at least 70%, 75%, 80%, 85%, 90%, 91%, 92%, 93%, 94%, 95%, 96%, 97%, 98% or 99% similarity to the amino acid sequence selected from any one of SEQ ID NOs: 3-5 and SEQ ID NOs: 10-12.

10. A fusion protein, comprising the engineered PSD protein according to claim 1.

11. The fusion protein according to claim 10, further comprising a sequence promoting the solubility of the PSD protein.

12. The fusion protein according to claim 11, wherein the sequence promoting the solubility of PSD protein is a GST sequence.

13. The fusion protein according to claim 10, further comprising one or more signal peptides.

14. A nucleic acid molecule, encoding the engineered PSD protein according to claim 1.

15. An expression vector, comprising the nucleic acid molecule according to claim 14.

16. An engineered cell, comprising the nucleic acid molecule according to claim 14 and a nucleic acid molecule encoding a cargo protein.

17. A method for preparing an engineered extracellular vesicle, wherein it comprises the following steps:
culturing the engineered cell according to claim 16, and isolating the extracellular vesicle secreted by the engineered cell from a culture medium.

18. An engineered extracellular vesicle, comprising the fusion protein according to claim 10 and a cargo protein.

19. A pharmaceutical composition, comprising the engineered extracellular vesicle according to claim 18, and a pharmaceutically acceptable carrier.

20. A method for diagnosing, treating and/or preventing a disease, comprising administrating to a subject in need thereof an effective amount of the engineered extracellular vesicle according to claim 18.
